(19) United States
(12) Patent Application Publication
(10) Pub. No.: US 2018/0228551 A1
(43) Pub. Date: Aug. 16, 2018

(54) BLADE POSITIONING DEVICE

(71) Applicant: GYRUS ACMI, INC., d/b/a Olympus Surgical Technologies America, Southborough, MA (US)

(72) Inventor: Riyad Moe, Madison, WI (US)

(21) Appl. No.: 15/749,328

(22) PCT Filed: Jun. 16, 2016
(86) PCT No.: PCT/US2016/037788

§ 371 Date: Jan. 31, 2018

Related U.S. Application Data
(60) Provisional application No. 62/222,466, filed on Sep. 23, 2015.

(51) Int. Cl. A61B 34/20; A61B 17/32

(52) U.S. Cl. CPC A61B 34/20; A61B 17/32002; A61B 2090/0811; A61B 2034/2059; A61B 2034/2051

(57) ABSTRACT

A medical instrument is disclosed. The medical instrument comprises a moveable tip, a mechanism for moving the tip, a surgical element on the tip; and a first sensing device. The first sensing device communicates one or more output signals to an image guidance system corresponding to movement in the mechanism so that a visual representation of the tip, the surgical element, or both is provided on a display.

### FIELD

These teachings relate generally to medical instruments, and more particularly to medical instruments for use with image guidance systems.

### BACKGROUND

Image guidance systems can provide a surgeon with a virtual representation of one or more medical instruments relative to pre-operative and/or intra-operative images of the anatomy and/or a surgical site. More specifically, image guidance systems may advantageously allow as surgeon to guide one or mare medical instruments through the anatomy, even though the medical instruments and/or the surgical site may not be within the surgeon's direct line of sight.

In image-guided surgery, generally, one or more pre-operative and/or intra-operative images of the anatomy and/or of the surgical site are acquired via computerized axial tomography (CAT scan), magnetic resonance imaging (MRI), or the like. An image guidance system is provided on or near the patient, and its position relative to the patient is recorded and preferably remains constant. One or more sensing devices are provided on one or more of the medical instruments. During use, the one or more sensing devices generate one or more output signals representative of the location of the one or more sensing devices, the one or more medical instruments, one or more portions of a medical instrument (e.g., a tip, a body, a surgical element, etc.), or a combination thereof. The output signals are communicated to the image guidance system, which also maintains the pre-operative and/or intra-operative images of the anatomy and/or of the surgical site. The image-guidance system provides a virtual representation on a display of the position, location, and/or orientation of the one or more medical instruments and/or the portions thereof relative to the pre-operative and/or intraoperative images. Thus, the surgeon can view, track, monitor, and/or guide the medical instruments relative to the surgical site, even though either or both may not be in the surgeon's line of sight.

As can be imagined, image guidance systems may be preferred in various procedures, such as minimally invasive and hard-to-reach medical procedures, like some sinus procedures. There remains a need, however, to improve and expand the types medical instruments used with image guidance systems. For example, some attempts have been made to provide a debrider for use with image guidance systems. Generally, a debrider is a medical instrument that has an outer blade and an inner blade located within the outer blade. During use, one or both of the blades are rotated and, via a cutting feature on one or both of the blades, various tissue, cartilage, bone, or the like can be shaved, cut, resected, abraded and/or removed. In order to use the debrider with an image guidance system, one or more sensing devices are typically provided near a distal end of the debrider. However, placement of one or more sensing devices near a distal end thereof may undesirably increase a section size of one or both of the blades. Moreover, some debriders are driven by an electric motor that produces magnetic fields, which can undesirably interfere with the sensing devices and/or the magnetic field generated by the image guidance system. Further, some debriders have electrosurgical capabilities that generate heat, which can undesirably interfere with sensing device functions when the sensing devices are located at or near a distal end of the debrider.

Accordingly, it may be desirable to provide a medical instrument for use with an image guidance system that overcomes at least one of the aforementioned challenges. For example, it may be attractive to have a method and/or a system for accurately determining the position of one or more blades of a medical instrument relative to each other, to the medical instrument, the body, a surgical site, etc., while also overcoming one of more of the aforementioned challenges. It may also be attractive to have a medical instrument that is part of a system that can determine the position of the outer blade; can determine the rotational orientation of one or more of the cutting windows of the blades; or both. It may be desirable to have a debrider for use with an image guidance system where the position of the outer blade, including its rotational orientation relative to the body, the anatomy, a surgical site, etc., can be accurately determined.

Some examples of debriders and positioning devices can be found in U.S. Pat. Nos. 8,702,702, 8,670,816 and U.S. Patent Application Publication Nos. 2013/0225943 and 2012/0101370, all of which are incorporated by reference herein for all purposes.

### SUMMARY

A medical instrument is disclosed that comprises a moveable tip; a mechanism for moving the tip; a surgical element on the tip; and a first sensing device. The first sensing device communicates one or more output signals to an image guidance system corresponding to movement in the mechanism so that a visual representation of the tip, the surgical clement or both is provided on a display.

A method is disclosed comprising obtaining the medical instrument according to the teachings herein; and moving the mechanism so that the tip moves. The first sensing device communicates the one or more output signals to the image guidance system corresponding to the movement in the mechanism so that the visual representation of the tip, the surgical element, or both is provided on the display.

A surgical device is disclosed comprising a body, a surgical element connected to the body, the surgical element being moveable relative to the body; a first sensing device connected to the body and restricted from moving relative to the body; and a mechanism for moving the surgical element relative to the body. The mechanism comprises a first member; and a second member, the second member being rotatable relative to the first member. The surgical device also comprises a second sensing device. The first sensing device generates a first output signal corresponding to a location or orientation of the body within a surgical environment and communicates the first output signal to an image guidance system. The second sensing device generates a second output signal corresponding to rotation of the second member relative to the first member and communicates the second output signal to the image guidance system so that a visual representation of a location of the surgical element relative to the surgical environment is provided on a display.

A surgical device is disclosed comprising a body; a first sensing device connected to the body, the first sensing device being fixed relative to the body; a surgical element connected to the body, the surgical element being moveable relative to the body; a first mechanism for moving the surgical element relative to the body. The first mechanism comprises a first mechanism first member; and a first mechanism second member, the second member being moveable relative to the first member. A second mechanism for moving the surgical element relative to the body, the second mechanism comprising a second mechanism first member; and a second mechanism second member, the second mechanism second member being moveable relative to the second mechanism first member. The surgical device also includes a third sensing device. The first sensing device generates a first output signal corresponding to a location of the body in a surgical environment and communicates the first output signal to an image guidance system. The second sensing device generates a second output signal corresponding to motion of or in the first mechanism and communicates the second output signal to the image guidance system. The third sensing device generates a third output signal corresponding to motion of or in the second mechanism and communicates the third output signal to the image guidance system. The image guidance system uses the first output signal, the second output signal, and the third output signal to provide a virtual representation of a location of the surgical element relative to the surgical environment on a display.

Also disclosed is a surgical device comprising a body; a first sensing device connected to the body, the first sensing device being unmovable relative to the body; a first surgical element connected to the body, the first surgical element being moveable relative to the body; a second surgical element connected to the body, the second surgical element being moveable relative to the body; and a first mechanism for moving the first surgical element relative to the body. The first mechanism comprising a first mechanism first member; and a first mechanism second member, the second member being moveable relative to the first member. The surgical device also includes a second sensing device, and a second mechanism for moving the second surgical element relative to the body. The second mechanism comprising a second mechanism first member; and a second mechanism second member, the second mechanism second member being moveable relative to the second member first member; and a third sensing device. The first sensing device generates a first output signal corresponding to a location of the body in a surgical environment and communicates the first output signal to an image guidance system. The second sensing device generates a second output signal corresponding to motion of or in the first mechanism and communicates the second output signal to the image guidance system. The third sensing device generates a third output signal corresponding to motion of or in the second mechanism and communicates the third output signal to the image guidance system. The image guidance system uses the first output signal, the second output signal, and the third output signal to provide a virtual representation of a location of the first surgical element and the second surgical element relative to the surgical environment on a display.

A surgical device is disclosed comprising a body, a surgical element connected to the body, the surgical element being moveable relative to the body, a mechanism for moving the surgical element relative to the body, the mechanism comprising: a first member; and a second member, the second member being rotatable relative to the first member, and a first sensing device connected to the first member; a second sensing device connected to the second member. The first sensing device generates and sends to an image guidance system a first output signal corresponding to a location or orientation of the first member in a surgical environment. The second sensing device generates and sends to the image guidance system a second output signal corresponding to a location or orientation of the second member in the surgical environment. The image guidance system uses the first output signal and the second output signal to provide a virtual representation of a location of the surgical relative to the surgical environment on a display.

A surgical device is disclosed, comprising a body; a removable module, comprising a surgical element; a mechanism for moving the surgical element relative to the removable module, the mechanism composing a first member; a second member that is moveable relative to the first member; a sensing device. The sensing device generates an output signal corresponding to movement in the mechanism.

Further disclosed is a surgical device, comprising a body; a removable module, comprising a surgical element that is moveable relative to the body; a mechanism for moving the surgical element relative to the; a mechanism for moving the surgical element relative to the removable module, the mechanism comprising; a first member that is moveable relative to the body; a second member that is moveable relative to the body and to the first member; a sensing device. The sensing device generates an output signal corresponding to movement of the second member relative to the first member.

Further yet, a surgical device is disclosed comprising a body and a surgical element that is moveable relative to the body. The surgical device also includes a mechanism, a first member, a second member, and a sensing device. The mechanism is configured to move the surgical element relative to the body. The first member is moveable relative to the body. The second member is movable relative to the first member and the body. The sensing device generates an output signal corresponding to movement of the second member relative to the first member in the mechanism.

A surgical device is disclosed comprising a body; a removable module, comprising a base element; a surgical element that is moveable relative to the base element; and a first sensing device connected to the base element, the first sensing device being unmovable relative to the base element. The first sensing device generates a first output signal corresponding to the location or orientation of the body, the base element, or both in a surgical environment and communicates the positional output signal to an image guidance system.

A surgical device is disclosed comprising a body; a removable module connected to the body, the removable module comprising: a surgical element that is moveable relative to the body; a mechanism for moving the surgical element, the mechanism comprising: a first member that is stationary relative to the surgical element; and a first sensing device attached to the first member, the first sensing device is unmovable relative to the first member. The first sensing device generates a first output signal corresponding to the location or orientation of the surgical element in a surgical environment and communicates the first output signal to an image guidance device.

### BRIEF DESCRIPTION OF THE DRAWINGS

FIG. 1 is a perspective view of a medical instrument.

FIG. 2 is a detailed perspective view of the cutting feature of the medical instrument of FIG. 1.

FIG. 3 is a detailed perspective view of a portion of the medical instrument of FIG. 1.

FIG. 4 is a detailed perspective view of a portion of the medical instrument of FIG. 1.

FIG. 5 is a detailed perspective view ala portion of the medical instrument of FIG. 1.

FIG. 6 is a perspective view of a medical instrument.

FIG. 7 is a perspective view of a medical instrument.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

This disclosure claims priority to U.S. Application No. 62/222,466 filed on Sep. 23, 2015, the entirety to which is hereby incorporated by reference herein for all purposes. The image guidance system can be any system where one or more medical instruments and/or portions of one or more medical instruments can be viewed, tracked, monitored, located, and or guided. Preferably, the image guidance system can be any suitable system where one or more medical instruments and/or portions of one or more medical instruments can be viewed, tracked, monitored, located, and/or guided. Preferably, the one or more medical instruments and/or portions of one or more medical instruments can be viewed, tracked, monitored, located, and/or guided in conjunction with or relative to one or more preoperative or intraoperative images. The image guidance system may provide for a surgeon or user to view, track, locate, monitor, and/or guide on a display one or more medical instruments relative to the anatomy and/or a surgical site or environment, even though either or both may not be directly in the surgeon's line of sight. The image guidance system can include a computer, logic system, or processor to read, receive, interpret, analyze, determine, or a combination thereof one or more signals that are sent from, or provided by, one or more sensing devices, encoders, sensors, magnets, the like, or a combination thereof. The image guidance system can be provided with any number of suitable signals in order to determine the location and/or position of one or more features (e.g., removable modules, mechanisms, tips, surgical elements, handles, bodies, etc.) of the medical instrument. The image guidance system can determine an error signal between two or more signals, or a deviation between two or more signals, to determine if the mechanism, the tip, the modules, the surgical element, or a combination thereof has been moved and/or to determine a location or position thereof.

The image guidance system, a computer, a processor, a logic system, the like, or a combination thereof can use one or more signals and/or other suitable information supplied by the one or more sensing devices, magnets, sensors, encoders, the like, or a combination thereof to provide and/or create a representation of the tip, the surgical element, the body, the medical instrument, or a combination thereof on a display. The representation can be a virtual image. The representation can be real-time, or the representation can be delayed. Preferably, the representation is real-time and is provided on the display in conjunction with one or more pre-operative and/or intra operative images of the anatomy, the surgical site, or both. The display can be located in a surgical room or the display can be located off-site outside of the surgical room. Preferably, the representation provides a virtual representation of the location of one or more portions of the medical instrument relative to the anatomy. The image guidance system may be of the type or similar to the system disclosed in U.S. Patent Application Publication number U.S. 2013/0225943A1, the disclosure of which is hereby incorporated by reference herein for all purposes.

The medical instrument can be any medical instrument. Preferably, the medical instrument is suitable for use with an image guidance system. The medical instrument may be capable of being held and manually manipulated by a surgeon or user during a procedure. The medical instrument can be placed on a stand or other holder and computer controlled. The medical instrument may be a debrider or a microdebrider. The medical instrument may be of the type disclosed in commonly owned U.S. Pat. No. 8,920,419 B2, filed on Mar. 14, 2013, the disclosure of which is hereby incorporated by reference herein in its entirety. Preferably, the medical instrument is a debrider that includes one or more blades or tubes, or, preferably, two or more blades or tubes. The medical instrument may be used to perform any suitable surgery. For example, the medical instrument can be used to perform a tonsillectomy, turbinoplasty, septoplasty, supraglottoplasty, sinus surgery, throat surgery, small joint arthroscopy, large joint arthroscopy, spinal surgery, disc surgery, the like, or a combination thereof.

The medical instrument may include one or more tips. The one or more tips may be used to perform a surgical procedure. The one or more tips may be integrally formed with the body, the medical instrument, or both, or may be securely attached thereto. The one or more tips may be removeably connected to the medical instrument, the body of the medical instrument, a handle, or a combination thereof. The one or more tips may be replaced or changed depending on the type of surgical procedure. For example, a larger tip or a smaller tip may be preferred depending on the specific type of procedure. Preferably, the one or more tips can be replaced before or after every surgical procedure. The one or more tips can be moved, removed, relocated, reoriented, repositioned, rotated, adjusted, bent, articulated, the like, or a combination thereof relative to the body, the medical instrument, or both. The one or more tips can be moved, relocated, reoriented, repositioned, rotated, bent, articulated, the like, or a combination thereof by manipulating one or more mechanisms or controls on the base, the medical instrument, a separate controller, the like, or a combination thereof. The one or more controls may include one or more buttons, knobs, sliders, collars, nose cones, levers, or a combination thereof. The one or more tips may be an end piece, a removable module or element, or both.

The one or more tips may include wiring so that the tip can be used with a monopolar energy source, a bi-polar energy source, free of energy, or a combination thereof. The one or more tips may be configured so that based upon the arrangement of the wires within the tip, the tip applies a monopolar energy, and/or a bi-polar energy to a procedure site. Alternatively, the one or more tips may be devoid of wiring for use with a mechanical tip, thereby the tip may not be configured to apply either monopolar or bipolar energy to a surgical site.

The one or more tips may include an outer blade or tube and an inner blade or tube, One or both of the blades can rotate and/or be rotated. One or both of the blades can rotate and/or be articulated. Preferably, during use, the inner blade can be rotated and the outer blade is substantially stationary. Preferably, the inner blade can be rotated with a motor and the outer blade is substantially stationary during use, but ran be rotated by rotating and/or otherwise manipulating a feature or control on the medical instrument, such as a mechanism or nosecone. The inner blade, the outer blade, or both may rotate clockwise, counterclockwise, or both. The inner blade may reciprocate or move back and forth. The outer blade may be rotatable so that a surgical element or cutting feature on the blade can be positioned and/or aligned to correspond to a surgical site or environment. For example, during a procedure, a surgeon may rotate the outer blade and the corresponding cutting blade opening so that the opening and/or the Cutting feature aligns with a surgical site or environment. Preferably, when the medical instrument is in the anatomy and/or out of the surgeon's line of sight, using the teachings disclosed herein, a surgeon can adjust the outer blade and the corresponding blade opening or cutting feature without having to remove the medical instrument from the anatomy and/or adjust the blade within the anatomy via trial-and-error.

The one or more tips can include one or more surgical elements. In some configurations, the one or more tips can preferably include two or more surgical elements. The surgical element can be any feature or combination of features for performing a surgical procedure. For example, the surgical element can be a cutting feature, a cutting window; an outer blade cutting window; an inner blade-cutting window; an electrode; a cryogenic delivery port; etc. The surgical element can be located on any portion and or at any location on the one or more tips. For example, the surgical element can be located on a proximal end of the tip, a distal end of the tip, or at a location therebetween. The surgical element can be located on any portion of the medical instrument. For example, the surgical element can be located on, at, or near the body; on, at, or near a proximal end of the medical instrument; on, at, or near a distal end of the medical instrument; or at any suitable location therebetween. The one or more surgical elements can be moveable relative to the medical instrument, a base, a body, a ground element, a removable module, or a combination thereof. In some configurations, when the removable module or the surgical element is assembled or otherwise connected to the body, the base does not move relative to the body.

The surgical element can be or include one or more cutting features. The one or more cutting features may be any feature that may cut, resect, shave, and/or remove tissue cartilage, bone or the like. The one or more cutting features may be located anywhere on the medical instrument. Preferably, the one or more cutting features are located at or near a distal end of the medical instrument. The cutting feature may comprise one or more openings in the outer blade, the inner blade, or both. One or both of the openings may include sharp and/or serrated teeth, edges, or the like. Rotating one or both of the blades may cause the cutting feature to cut, resect, shave, and/or remove tissue cartilage, bone, or the like.

The medical instrument can include one or more mechanisms. The one or more mechanisms may be moved or otherwise manipulated so that the tip, the one or more blades, the one or more surgical elements, or a combination thereof move, relocate, reorient, reposition, rotate, articulate, bend, the like, or a combination thereof relative to the body, the medical instrument, or both. The one or more mechanisms can be or can include one or more links, bodies, members, dials, gears, sliders, nose cones the like, or a combination thereof. The one or more mechanisms can include a ground link, one or more base elements, base links, or a combination thereof. The one or more base elements may function to connect to the body or the hand piece. Preferably, the one or more base elements do not move relative to the tip. The one or more base elements may include a base link providing half of a journal bearing for a spinning nose cone. Accordingly, the journal bearing is located within the tip and therefore would not be required to withstand repeated sterilization. The one or more sensing devices, sensors, magnets, encoders, etc. may be provided on the base link, the base element, or both. The one or more mechanisms, links, bodies, members, etc. can be rigidly connected to the body, the one or more surgical elements, or both. In some configurations, the one or more members, links, bodies, members, etc. are not rigidly connected to the body, the one or more surgical elements, or both. The one or more mechanisms, links, bodies, members, etc. can be removably connected to the body, the surgical elements, or both. The one or more mechanisms can be moved or manipulated so that an outer tube or blade is rotated so that the outer blade cutting window can be oriented; an inner blade is rotated so that the inner blade cutting window rotates; or both. The one or more mechanisms can be moved or otherwise manipulated. For example, the one or more mechanisms and, therefore, the surgical element, can be moved by moving, sliding, translating, rotating, or otherwise manipulating a suitable switch, button, knob, link, control, nose cone, wheel the like, or a combination thereof. The one or more mechanisms can be controlled by the surgeon or user holding the medical instrument, or the mechanism can be controlled by a remote user viewing the display of the image guidance system, for example. One or more sensing devices, sensors, encoders, magnets, systems, can be used to sense movement of the one or more mechanisms, members, bodies, links, etc. etc.

The medical instrument may include one or more sensing devices. The one or more sensing devices may cooperate with die image guidance system so that a surgeon can view, track, monitor, locate, and/or guide one or more medical instruments through the anatomy even though the medical instrument and/or the anatomy may not be in the surgeon's line of sight. The one or more sensing devices may cooperate with the image guidance system to provide a relative location, position, and/or orientation of the medical instrument, the one or more blades, the tip, the one or more surgical elements, or a combination thereof relative to the medical instrument, the anatomy, a site of interest in the anatomy, or a combination thereof. The one or more sensing devices may determine or read the relative position or speed of a rotating or reciprocating inner blade or tube, outer tube or blade, or both. The one or more sensing devices may determine or read the relative position or speed of the motor configured to rotate one or more of the blades; determine or read the relative orientation of the outer blade cutting window relative to the inner blade cutting window; or a combination thereof. The one or more sensing devices may sense relative movement between one or more mechanisms. Advantageously, the one or more sensing devices may cooperate with an image guidance system so that a surgeon can move or rotate the outer blade and the corresponding surgical element arid/or cutting blade opening to align with a surgical site without having to remove the medical instrument from the anatomy and rotate the blade, for example. The one or more sensing devices may sense relative movement and for a position or location of one or more controls (e.g., knobs, switches, buttons, nose cones sliders, etc.) relative to the base, hand piece, tip, medical instrument, or a combination thereof, and determine a relative location and/or position of the tip, one or more surgical elements, removable modules, or a combination thereof. For example, the one or more sensing devices may sense movement, rotation, and/or manipulation of the nosecone, and determine the rotational position of the outer blade or tube that is moved or rotated by the nosecone. The one or more sensing devices may comprise any suitable number and/or any suitable type of sensors, magnets, encoders, or a combination thereof. For example, the one or more sensors may be electromagnetic sensors, electromagnetic coils, digitizers, optical tracking sensors, visual sensors, image guidance system (IGS) sensors, the like, or a combination thereof. The one or more sensors may sense one or more degrees of freedom, two or more degrees of freedom, three or more degrees of freedom, four or more degrees of freedom, or even five or more degrees of freedom (e.g., six degrees of freedom or more). The one or more sensors may determine an X, Y, Z position, an I, J, K, position, or any combination of positions. The one or more sensing devices may communicate the one or more output signals to an image guidance system, a device, computer, console, logic system, processor, etc. to read and/or interpret the one or more output signals. For example, the one or more output signals may be communicated wirelessly, or via one or more flying leads connected directly to the image guidance system, console, etc.

The one or more sensing devices, sensors, encoders, magnets, or a combination thereof may be provided or attached to any portion of the medical instrument. Preferably, the one or more sensing devices, sensors, encoders, magnets, or a combination thereof are provided or attached at a location on the medical instrument where the sensors do not contact the anatomy; do not increase a section size of the medical instrument, the one or more blades, or a combination thereof; are at a reduced risk of shorting or being heated; or a combination thereof. In some configurations, the one or more sensing devices, sensors, encoders, magnets, or a combination thereof may be provided on or in the body of the medical instrument and separate from the tip. The one or more sensing devices, sensors, encoders, magnets, or a combination thereof may be provided on or attached to one or more mechanisms, links, bodies, members, etc.; near one or more mechanisms, link, bodies, members, etc., or a combination thereof. The one or more sensing devices, sensors, encoders, magnets, or a combination thereof may be provided on or in the nosecone so that movement or rotation of the nosecone can be sensed and correlated to movement or rotation of one or more surgical elements, removable modules, etc. In this regard, advantageously, the one or more sensing devices, sensors, encoders, magnets, or combination thereof do not have to be discarded after the tip is set aside, replaced, or discarded, for example. This may reduce waste tidier the cost of the one or more interchangeable tips. In some configurations, the one or more sensing devices, sensors, encoders, magnets, or a combination thereof may be provided on or in the tip, the inn blade, the outer blade, and/or on one or more mechanisms, members, links, elements, etc. that may be used to move, relocate, and/or reposition the tip and/or one or more of the surgical elements. The one or more sensing devices, sensors, encoders, magnets, or a combination thereof may be located on a ground link, near a proximal end of the tip, such as where the tip contacts the body of the medical instrument, for example. The one or more sensing devices, sensors, encoders, magnets, or a combination thereof can be located on the medical instrument and their location or position relative to a surgical element, a distal end of the tip, the one or more cutting windows, outer blade cutting window, or a combination thereof is preferably known and remains fixed. Accordingly, the image guidance system can easily determine a location or position of the one or more sensing devices, sensors, encoders, magnets, or a combination thereof and then calculate and/or determine a location or position of the one tip, one or more surgical elements, or both.

The one or more magnets may be permanent magnets, electromagnets, or both. The or more encoders may be linear encoders, rotational encoders, or both. The encoder can be a rotational potentiometer. The encoder can pass a DC voltage. The encoder may be a linear encoder, a rotational encoder, or a combination thereof. The encoder may of the type described U.S. Pat. No. 8,670,816, the disclosure of which is hereby incorporated by reference herein for all purposes.

During use, the image guidance system can generate one or more magnetic fields. The magnetic field generated by the image guidance system induces Eddy currents in the one or more sensing devices, sensors, or both; changes an electrical current and/or a magnitude of electrical current in the one or more sensing devices, sensors, or both; or a combination thereof. Information relating to or corresponding to one or more of the aforementioned effects of the magnetic field on the one or more sensing devices, sensors, or both an be communicated to the image guidance system via one or more output signals. Alternatively, or in addition, the one or more sensing devices, sensors, or both may perturb a magnetic field generated by the image guidance system, which can be communicated to the image guidance system in the same or via different output signals. From the one or more output signals, the image guidance system can calculate and/or determine the location and/or position of the distal end of the medical instrument, the tip, the surgical elements, the one or more cutting windows, the outer blade cutting window, or a combination thereof. More specifically, because the location of the one or more sensors relative to the location and/or position of the distal end of the medical instrument, the one or more surgical elements is known, once the location of the sensor is determined, the location and/or position of the distal end of the medical instrument, the one or surgical elements can be calculated and/or determined.

The one or more magnets may function to provide a relative location, position, and/or orientation of the medical instrument, the one or more blades, the tip, the surgical elements, or a combination thereof. Preferably, the one or more magnets cooperate with the image guidance system, the one or more sensors or sensing devices, or both to provide a rotational position or location of the tip, a distal end of the outer blade, the surgical elements, or a combination thereof. The one or more magnets may be provided or attached to any portion of the medical instrument.

The one or more magnets may induce Eddy currents in the one or more sensors or sensing devices, which can be communicated to the image guidance system. The one or more magnets may perturb a magnetic field generated by the image guidance system, which can be communicated to the image guidance system. Information relating to or corresponding to the output signal, the induced eddy currents, the perturbed magnetic field, or a combination thereof can be communicated to the image guidance system via the one or more output signals from the sensors or via different signals.

FIG. 1 illustrates a medical instrument 10. The medical instrument 10 includes a body 12 and a tip 14. The tip 14 includes an outer blade 16 and an inner blade 18 that is located within the outer blade 16. The medical instrument 10 includes a nosecone 20 for rotating the outer blade 16. The medical instrument 10 includes a motor 22 for rotating the inner blade 18. The medical instrument 10 includes a connection enclosure 24. The medical instrument 40 includes a mechanism 26 for relocating, repositioning, and/or reorienting the tip 14 relative to the body 12, a site of interest in the anatomy, or both. The tip 14 includes a surgical element 28, which may be a cutting feature located at or near a distal end thereof. The tip 14 is angled between a proximal end and a distal end.

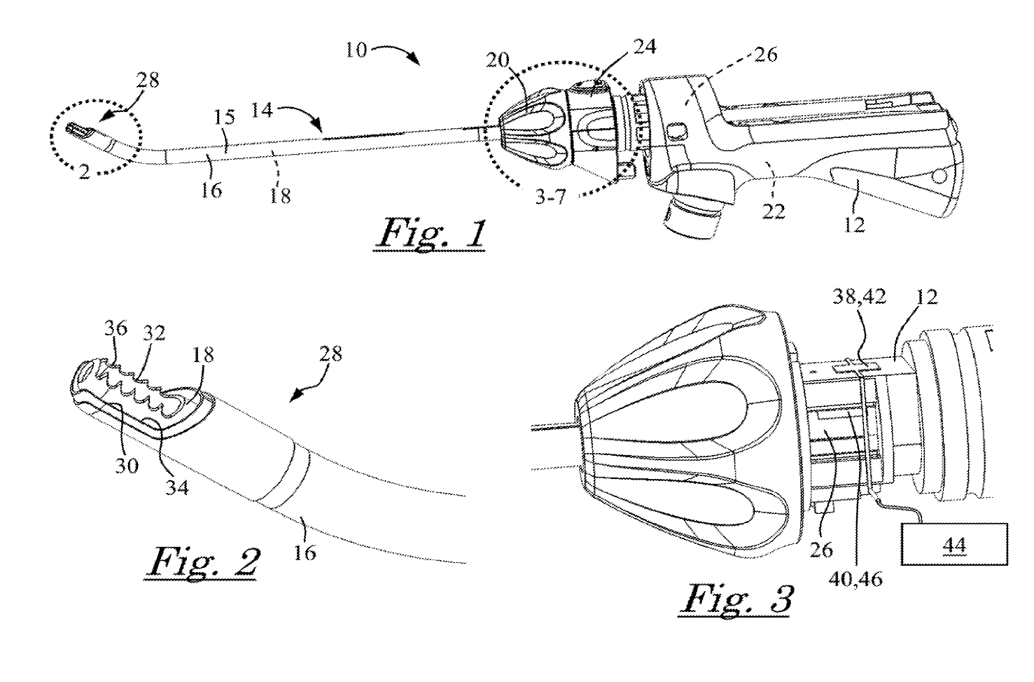

FIG. 2 is a detailed view of the surgical element 28 of FIG. 1. The surgical element 28 or cutting feature includes an outer blade cutting window 30 on the outer blade 16, and an inner blade cutting window 32 on the inner blade 18. The outer blade cutting window 30 includes outer window cutting edges 34. The inner blade cutting window 32 includes inner window cutting edges 36.

Referring to both FIGS. 1 and 2, during use, the motor 22 rotates the inner blade 18 so that the cutting blade windows 28, 30 become aligned with each other (as shown in FIGS. 1 and 2) and then become misaligned with each other knot shown). While rotating, tissue, cartilage, bone, and/or the like can be shaved, cut, resected, abraded and/or removed via the cutting blade windows 28, 30 and/or the window edges 30, 32. The nosecone 20 can be rotated to rotationally adjust the outer blade 16 and/or the outer blade cutting window 30 relative to the inner blade 18, the inner blade cutting window 32, the body 12, a site of interest in the anatomy, or a combination thereof.

FIG. 3 is a detailed view of a portion of the medical instrument 10 of FIG. 1 connection enclosure 24 removed for clarity. The medical instrument 10 includes a first sensing device 38 and a second sensing device 40. The first sensing device 38 includes a first sensor or sensing device 42, which may be an electromagnetic image guiding system sensor, a visual image guiding system sensor, the like, or a combination thereof. The first sensor 42 is attached to a portion of the body 12. The first sensor 42 is in communication with an image guiding system 44. The image guidance system 44 can generate a magnetic field. The second sensing device 40 includes a second sensor or sensing device 46, which may be a rotational encoder. The second sensor 46 is also in communication with an image guiding system 44.

With reference to FIGS. 1-3, during use, the first sensor 42 is configured to read the magnetic field generated by the image guidance system 44 and send one or more corresponding output signals to the image guidance system 44 corresponding to the location of the first sensor 42, the medical instrument 10, the body 12, or a combination thereof. The location of the first sensor 42, the medical instrument 10, the body 12, or a combination thereof may be relative to a surgical environment, such as the operating room, a patient, a surgical site in anatomy, or a combination thereof.

During use, one or more controls, the nosecone 20, or both can be manipulated and/or rotated so that the mechanism 26, one or more elements in the mechanism 26 (e.g., links, bodies, etc.), or a combination thereof move and, therefore, relocate, reposition, articulate, and/or reorient the tip 14, the outer blade 16, or both. The second sensor 46 senses relative movement in the mechanism 26, or between the elements in the mechanism 26, and sends a corresponding output signal to the image guiding system 44. The image guidance system 44 combines the output signals from the first sensor 42 and the second sensor 46 to create a visual display or representation of the tip 14, the outer blade 16, and/or the outer blade cutting window 28 relative to the body 12, site of interest in the anatomy, or both on a display.

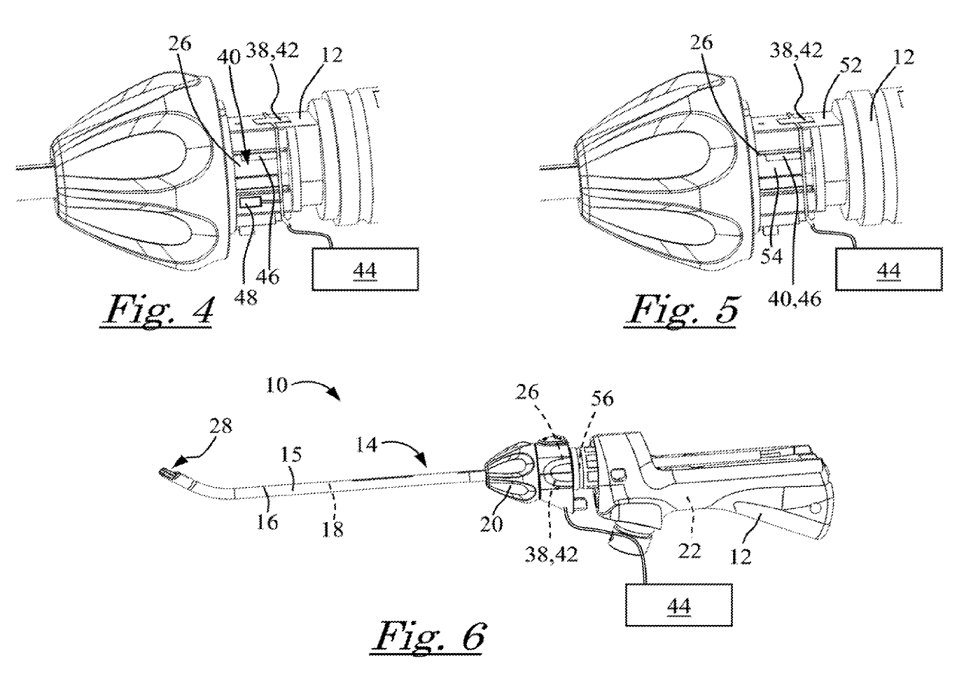

FIG. 4 is a detailed view of a portion of the medical instrument 10 of FIG. 1 with the connection enclosure 24 removed for clarity. The medical instrument 10 includes a first sensing device 38 and a second sensing device 40. The first sensing device 38 includes a first sensor or sensing device 42 that may be an electromagnetic image guiding system sensor, a visual image guiding system sensor, or both. The first sensor 42 is attached to a portion of the body 12. The first sensor 42 is in communication with an image guiding system 44. The image guidance system 44 can generate a magnetic field. The second sensing device 40 includes a second sensor or sensing device 46 and a third sensor or sensing device 48, both of which may be rotational encoders, linear encoders, or a combination thereof. The second and third sensors 46, 48 are in communication with the image guiding system 44.

With reference to FIGS. 1, 2, and 4, during use, the first sensor 42 is configured to read the generated magnetic field from the image guidance system 44 and send a corresponding signal to the image guidance system 44 of the location of the medical instrument 10, the body 12, or both. During use, one or more controls, the nosecone 20, or both can be manipulated and/or rotated so that the mechanism 26, one or more elements in the mechanism 26 (e.g., links, bodies, etc.), or a combination thereof move and, therefore, relocate, reposition, articulate, and/or reorient the tip 14, the outer blade 16, or both. The controls, the nosecone 20, or both can be used to move the tip 14 through a swept arc in space. During use, the second and third sensors 46, 48 can sense relative movement, a position, or motion in the mechanism 26, between the elements, between one or more elements relative to one or more other elements, or a combination thereof and send corresponding signals to the image guidance system 44. The image guidance system 44 can use or combine the signals from the first sensor 42 and the second and third sensors 46, 48 to create a visual real time location/position of the tip 14, the outer blade 16, and/or the outer blade cutting window 28 relative to the body 12, site of interest in the anatomy, a surgical environment, or a combination thereof, on a display.

With continued reference to FIG. 4, in another configuration, the medical instrument 10 of FIG. 1 is illustrated with the connection enclosure 24 removed for clarity. The medical instrument 10 includes a first sensing device 38, a second sensing device 40, and a third sensing device 48, all three of which are in communication with the image guidance system 46. The first sensor 42 is attached to a portion of the body 12.

With continued reference to FIGS. 1, 2, and 4, in another configuration, during use, the first sensor 42 is configured to read the generated magnetic field from the image guidance system 44 and send a corresponding signal to the image guidance system 44 of the location of the medical instrument 10, the body 12, or both. During use, the first mechanism can be manipulated, moved, and/or rotated so that the first surgical element (e.g., the tip 14, the outer blade 16, the outer blade eating window, etc.) moves. The movement of the first mechanism can be detected by the second sensing device 38, which generates and sends a corresponding output signal to the image guidance system 46. The second mechanism can also be manipulated, moved, and/or rotated so that the second surgical element (e.g., the tip 14, the outer blade 16, the outer blade cutting window, etc.) moves. The movement of the second mechanism can be detected by the third sensing device 48, which generates and sends a corresponding output signal to the image guidance system 46. The image guidance system 46 can use the first, second, and third output signals to provide a virtual representation of the location of the fast surgical element, the second surgical element, the body, or a combination thereof on a display.

FIG. 5 is a detailed view of a portion of the medical instrument 10 of FIG. 1 with the connection enclosure 24 removed for clarity. The medical instrument 10 includes a first sensing device 38 and a second sensing device 40. The first sensing device 38 includes a first sensor or sensing device 42 and the second sensing device 40 includes a second sensor or sensing device 46. The first sensor 42, the second sensor 46, or both, may be an electromagnetic image guiding system sensor, a visual image guiding system sensor, or both. The first sensor 42 and the second sensor 46 are in communication with an image guiding system 44. The image guidance system 44 can generate a magnetic field. The mechanism 26 includes a first member 52 and a second member 54. Neither the first member 52 nor the second member 54 is rigidly connected to the body 12 or the surgical element 28. The first sensor 42 is attached to the first member 52, and the second sensor 46 is attached to the second member 54.

With reference to FIGS. 1, 2, and 5, before use, the sensors 42, 46 can send one or more baseline signals to image guiding system 44. The baseline signals can represent an in or baseline position before any movement of the first and second members 52, 54, the mechanism 26, the tip 14, the outer blade 16, and/or the outer blade cutting window 28 relative to the body 12, site of interest in the anatomy, or both. During use, one or more controls, the nosecone 20, or a combination thereof can be manipulated and/or moved so that the first and second members 52, 54 move and therefore relocate, reposition, and/or reorient the tip 14, the outer blade 16, and/or the outer blade cutting window 28. While the members 52, 54 move, the sensors 42, 46 send additional signals corresponding to the relative position, motion, and/or movement between the members 52, 54, relative to other members, relative to the body, the surgical environment, or a combination thereof to the image guiding system 44. The image guidance system 44 compares the initial, baseline signals to the additional signals and determines a deviation therebetween. The deviation represents how far the first member 52, the second member 54, and/or the mechanism 26 has moved from the initial, baseline position. From this deviation, the image guidance system 44 can create a visual, real time location/position of the tip 14, the outer blade 16, and/or the outer blade cutting window 28 relative to the body 12, site of interest in the anatomy, or both on a display.

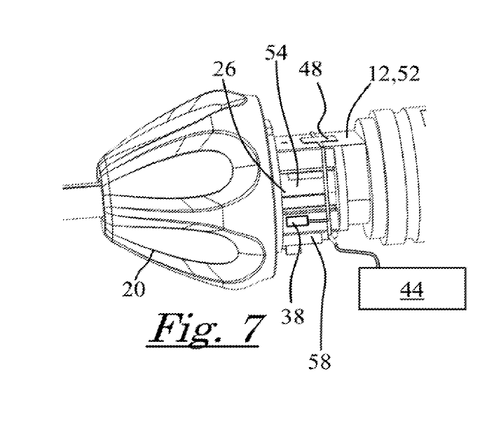

FIG. 6 illustrates an example of a medical instrument 10. The medical instrument 1 includes a body 12 and a removable module that may be a tip 14. The removable module or tip 14 includes an outer blade 16 and an inner blade 18 located within the outer blade 16. The medical instrument 10 includes a mechanism that may be a nosecone 20 that rotates the outer blade 16, and a motor 22 fear rotating the inner blade 18. The medical instrument 10 also includes a mechanism 26 for relocating, repositioning, and/or reorienting the tip 14 with respect to the body 12. The mechanism 26 includes one or more members 56 that move relative to each other in order to move, relocate, reposition, and/or reorient the tip 14, the outer blade 16, the inner blade 18, and/or the blade cutting windows (FIG. 2). A first sensing device 38 is contained in the removable module or tip 14 that reads or interprets relative motion, movement or a position of the mechanism 56 the relative movement, motion or position between the one or more members 56 (i.e., of the second member relative to a first member or any other member or link, or vice versa); the relative position or speed of the inner blade 18 or outer reciprocating blade 16; the outer blade window relative to the inner blade window (FIG. 2); or a combination thereof. The first sensing device 38 can read or interpret the speed of the motor, or the orientation of the outer blade window relative to the inner blade or inner blade window when the inner blade is not moving. The first sensing device 38 includes a first sensor 42, which may be an encoder. An output signal from the first sensor 42 is generated and sent to the image guiding system 46. From the output signal from the first sensor 42, the image guiding system 46 can display a visual location/position of the tip 14, the outer blade 16, and/or the outer blade cutting window 28 on a display. In some configurations, the removable module or tip 14 may also include a second sensing device and/or a second sensor that may be an electromagnetic image guiding system sensor, a visual image guiding system sensor, or both. Similarly, the second sensing device, the second sensor, or both can read the relative position between the one or more members 56; the relative position or speed of the inner blade 18 or outer reciprocating blade 16; the outer blade window relative to the inner blade window (FIG. 2); or a combination thereof. The second sensing device can read or interpret the speed of the motor, or the orientation of the outer blade window relative to the inner blade or inner blade window when the inner blade is not moving, or both. An output signal from the second sensing device and/or second sensor 42 can be also provided to the image guidance system 44 to create a visual, real time location of the tip 14, the outer blade 16, and/or the outer blade cutting window 28.

FIG. 7 illustrates an example of a surgical device or medical instrument 10. The medical instrument 10 includes a body 12 and a removable module that may be a tip 14 (see FIG. 6, for example). The removable module or tip 14 includes an outer blade 16 and an inner blade 18 located within the outer blade 16. The medical instrument 10 includes a mechanism that may be a nosecone 20 that can be manipulated to rotate the outer blade 16. The medical instrument 10 also includes a mechanism 26 for relocating, repositioning, and/or reorienting the removable module or tip 14 or surgical element 28 (FIG. 1) with respect to the body 12. The mechanism 26 includes a first member 52, a second member 54, and a base element 58. The base element 58 may connect the removable module or tip 14 to the body or hand piece 12. The surgical element 28 is movable relative to the base clement 58. A first sensing device 38 is connected to the base element 58 and is preferably unmovable relative to the base element 58. The first sensing device 38 generates an output signal corresponding to the location or orientation of the body 12, the base element 58, or both relative to a surgical environment. The second member 54 is rotatable relative to the first member 52. A second sensing device 48 generates an output signal corresponding to a position of the first member 52 relative to the second member 54. The output signals from the first and second sensing devices 38, 48 are communicated to an image guidance system 44 so that a visual representation of the surgical device or medical instrument 10 can be provided on a display.

With continued reference to FIG. 7, in another configuration, the surgical device or medical instrument 10 includes a body 12 and a removable module that may be a tip 14 (see FIG. 6, for example). The removable module or tip 14 includes an outer blade 16 and an inner blade 18 located within the outer blade 16. The medical instrument 10 includes a mechanism that may be a nosecone 20 that can be manipulated to rotate the outer blade 16. The medical instrument 10 also includes a mechanism 26 for relocating, repositioning, and/or reorienting the removable module or tip 14 or surgical element 28 (FIG. 1) with respect to the body 12. The mechanism 26 includes a first member 52, a second member 54, and a base element 58. The base element 58 may connect the removable module or tip 14 to the body or hand piece 12. The surgical element 28 is movable relative to the base element 58. A first sensing device 38 is connected to the base element 58 and is preferably unmovable relative to the base element 58. The first sensing device 38 generates an output signal corresponding to the location or orientation of the surgical element 28 in a surgical environment. The output signal from the first sensing device 38 is communicated to an image guidance system 44 so that a visual representation of the surgical device or medical instrument 10 can be provided on a display.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the Teachings of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission m the following claims of any aspect of subject matter that is disclosed herein is not a disclaimer of such subject matter, nor should it be regarded that the inventors did not consider such subject matter to be part of the disclosed inventive subject matter.

1)-16) (canceled)

17) A medical instrument, comprising:
i. a moveable tip;
ii. a mechanism for moving the tip;
iii. a surgical element on the tip;
iv. a first sensing device;
v. a body; and
vi. a second sensing device;
wherein the first sensing device communicates one or more output signals to an image guidance system corresponding to movement in the mechanism so that a visual representation of the tip, the surgical element, or both is provided on a display,
wherein the body is attached to the tip, and
wherein the second sensing device communicates one or more output signals to the image guidance system corresponding to a location or position of the body so that the image guidance system creates the visual representation of the body relative to the tip, a surgical site, or both.

18) (canceled)

19) (canceled)

20) The medical instrument of claim 17, wherein the mechanism includes a first member and a second member,
wherein the first sensing device includes a first sensor attached to the first member and a second sensor attached to the second member,
wherein the first and the second sensors each send a baseline signal to the image guidance device representing an initial position of the mechanism before the mechanism is moved, the fast and the second sensors each send additional signals to the image guidance device representing relative movement between the first member and the second member when the mechanism is moved, and
wherein the image guidance device determines a deviation between the signals to provide the visual representation of the tip, the surgical element, or both based on the deviation.

21) The medical instrument of claim 20 wherein the first sensor and the second sensor are an electromagnetic image guidance system sensor, a visual image guidance system sensor, or both.

22) The medical instrument of claim 17, wherein the mechanism includes two or more members, and
wherein the first sensing device includes a first encoder that reads linear motion, rotational motion, or both between the members when the mechanism is moved and
wherein information relating to the linear motion, the rotational motion, or both between the members is included in the one or more output signals.

23) The medical instrument of claim 22, wherein the first sensing device includes a second encoder,
wherein the second encoder reads linear motion, rotational motion, or both between the members when the mechanism is moved, and
wherein information relating to the linear motion, the rotational motion, or both between the members is included in the one or more output signals.

24) The medical instrument of claim 22 wherein the first encoder is provided on the first member,
wherein the first member moves with respect to the second member when the mechanism is moved, and
wherein the first sensor senses a relative position of the first member relative to the second member which is included in the one or more output signals.

25) The medical instrument of claim 17, wherein the tip is separable from the body, and
wherein the first sensing device is contained in the tip.

26) The medical instrument of claim 17, wherein the tip is separable from the body, and
wherein the first sensing device is contained in the body.

27) The medical instrument of claim 17, wherein the tip comprises:
i. an outer blade; and
ii. an inner blade at least partially disposed within the outer blade.

28) The medical instrument of claim 17, wherein the surgical element is an outer blade cutting window.

29) (canceled)

30) A surgical device comprising:
a body,
a surgical element connected to the body, the surgical element being moveable relative to the body,
a mechanism for moving the surgical element relative to the body, the mechanism comprising:
i. a first member; and
ii. a second member, the second member being movable relative to the first member, and
a first sensing device connected to the first member;
a second sensing device connected to the second member;
wherein the first sensing device generates and sends to an image guidance system a first output signal corresponding to a location or orientation of the first member in a surgical environment,
wherein the second sensing device generates and sends to the image guidance system a second output signal corresponding to a location or orientation of the second member in the surgical environment, and
wherein the image guidance system uses the first output signal and the second output signal to provide a virtual representation of a location of the surgical relative to the surgical environment on a display.

31. The surgical device of claim 30, wherein the first sensing device comprises an electromagnetic sensor, a visual sensor, or both.

32. The surgical device of claim 31, wherein the second sensing device comprises an electromagnetic sensor, a visual sensor, or both.

33) The surgical device of claim 30, wherein the surgical element is not separable from the body.

34) The surgical device of claim 30, wherein the first sensing device, the second sensing device, or both is a six-degree of freedom sensor.

35) The surgical device of claim 30, wherein the surgical element comprises an inner blade window.

36)-40) (canceled)

41) A surgical device comprising
i. a body;
ii. a surgical element, the surgical element being moveable relative to the body; and
iii. a mechanism for moving the surgical element relative to the body, the mechanism comprising:
a) a first member, the first member being moveable relative to the body;
b) a second member, the second member being movable relative to the first member and the body;
c) a sensing device,
wherein the sensing device generates an output signal corresponding to movement of the second member relative to the first member in the mechanism.

42) The surgical device of claim 41, wherein the surgical element is moveable relative to the first member and the second member.

43) The surgical device of claim 41, wherein the surgical element comprises an inner blade window.

44) The surgical device of claim 41, wherein the surgical element comprises an outer blade window.

45)-53) (canceled)
